(19) United States
(12) Patent Application Publication
(10) Pub. No.: US 2024/0032543 A1
(43) Pub. Date: Feb. 1, 2024

(54) ANTIMICROBIAL AND ANTIVIRAL NANOCOMPOSITES SHEETS

(71) Applicant: Claros Technologies Inc., St. Paul, MN (US)

(72) Inventors: Abdennour Abbas, Falcon Heights, MN (US); Andrew Elias Gonzalez, St. Paul, MN (US); John Wilfrid Brockgreitens, Minneapolis, MN (US); Michelle Marie Bellanca, Stillwater, MN (US)

(73) Assignee: Claros Technologies Inc., St. Paul, MN (US)

(21) Appl. No.: 18/256,685

(22) PCT Filed: Dec. 10, 2021
(86) PCT No.: PCT/US2021/062766

§ 371 Date: Jun. 9, 2023

Related U.S. Application Data
(60) Provisional application No. 63/123,814, filed on Dec. 10, 2020.

(51) Int. Cl. A01N 59/16; A41D 13/11; D06M 16/00; D06M 11/44; A01N 25/34; A01P 1/00; A62B 23/02; A41D 31/30

(52) U.S. Cl. CPC A01N 59/16; A41D 13/1192; D06M 16/00; D06M 11/44; A01N 25/34; A01P 1/00; A62B 23/025; A41D 31/305; D06M 2101/06

(57) ABSTRACT

Antimicrobial textiles and methods of making antimicrobial textiles including a sheet substrate comprising a textile and metal oxide nanoparticles in which the nanoparticles are present as a nanocomposite on the surface of and within the sheet substrate. The textiles may be used in wearable items such as personal protective equipment such as face masks. Methods of making the textiles include applying a metal salt solution to a textile to diffuse the metal salt into the textile and drying the textile, such as drying the textile with heat, to bind the metal salt to the surface of and the interior fibers of the textile by forming a nanocomposite of metal nanoparticles or nanostructures in situ.

### BACKGROUND OF THE INVENTION

Antimicrobial compositions are widely used for preventing the spread of infection. Antimicrobial compositions may be applied to or incorporated into surfaces that are frequently touched such as counter tops. In some cases, it may be desirable to functionalize materials with other compositions to give the materials antimicrobial properties. Textiles are popular materials for antimicrobial functionalization.

Antimicrobial textiles may be created by coating the textile with an antimicrobial agent. Such methods may produce a superficial antimicrobial surface which may not penetrate the fabric. In addition, the antimicrobial properties of the textile may not last through repeated laundry cycles. In some cases, chemical additives may be added to laundry wash cycles to give the textile qualities such as resistance to fouling or bacterial resistance. However, these additives may be released into the water stream at the end of the laundry cycle and may have a negative impact on the environment. In addition, it is often preferable that such antimicrobial functionalization does not change the appearance or quality of the textile and that it be durable during and after laundering, storage, and use. If the textile is in contact with skin, it is also preferable that it does not cause any irritation or adverse reaction.

In some cases, airborne microbes may be more difficult to manage than surface microbes. Airborne microbes are often controlled through air flow and air filtration mechanisms. For example, surgical suites and certain patient rooms may have double door systems and negative pressure to control air flow. Air filters may be used, and medical staff may wear masks over their noses and mouths as a physical barrier to filter microbes from being inhaled by the wearer or exhaled into the environment. Microbes become trapped in the masks as the air flows through them so that, hopefully, the medical staff do not become infected and do not pass infection to others. However, such masks have limitations. Depending upon their porosity, masks may still allow some microbes to pass through. In addition, the masks may become saturated as they collect airborne particles, reducing their usefulness and useful shelf life, resulting in the need for more frequent replacement. Furthermore, since the masks function by trapping airborne microbes, the masks themselves become a hazard that can spread infection. Some microbes such as COVID-19 remain stable for long periods of time, such that the masks themselves risk contaminating medical staff and other patients. As such, while these masks function as reducing the spread of infection, they still have limitations and there is a need for further improvement.

Therefore, while it is desirable to functionalize materials such as textiles to provide antimicrobial properties, improved methods of functionalization are needed to enable large scale production of such textiles, to improve their characteristics, to improve performance, to reduce the environmental impacts of the process, and to allow for broader use of antimicrobial functionalized materials in antimicrobial products to reduce the spread of infections, particularly in patient care settings.

### BRIEF DESCRIPTION OF THE DRAWINGS

While the specification concludes with claims particularly pointing out and distinctly claiming the subject matter that is regarded as forming the various embodiments of the present disclosure, it is believed that the disclosure will be better understood from the following description taken in conjunction with the accompanying Figures, in which:

FIG. 1 is an example of an antibacterial adhesive nanocomposite film according to various embodiments;

FIG. 2 is an example of an antimicrobial face mask according to various embodiments;

FIG. 3 is an example of an antimicrobial tampon according to various embodiments;

FIG. 4 is an example of an antimicrobial sanitary napkin according to various embodiments;

FIG. 5 is an example of an antimicrobial wound care pad according to various embodiments;

FIG. 6 is an example of a commercial dryer according to various methods;

FIG. 7 is another example of a commercial dryer according to various methods;

FIG. 8 is another example of a commercial dryer according to various methods;

FIGS. 9A and 9B are Scanning Electron Microscope (SEM) photographs of an untreated textile;

FIGS. 10A and 10B are SEM photographs of a treated textile according to various embodiments;

FIGS. 11 is a photograph of experimental results of antibacterial testing of nanocomposite textile tested using Pseudomonas aeruginosa;

FIG. 12 is a photograph of experimental results of antibacterial testing of nanocomposite textile tested using Staphylococcus aureus;

FIG. 13 is a photograph of experimental results of antibacterial testing of nanocomposite textile tested using Pseudomonas aeruginosa and Staphylococcus aureus;

FIG. 14 is a set of SEM photographs of zinc-polyurethane nanocomposite film (A) and zinc-nylon composite film (b) according to various embodiments verses controls;

FIG. 15 is set of SEM photographs of zinc-aramid nanocomposite textile fibers (a), silver-polyester nanocomposite textile fibers (b) and iron-polyurethane nanocomposite textile fibers (c) according to various embodiments;

FIG. 16 are X-ray Diffraction spectra of TiO2 nanoparticles (a) and ZnO nanoparticles obtained from nanocomposite textiles according to various embodiments;

FIG. 17 is EDS-SEM data of ceramic nanoparticles on the surface of an aramid fiber (a), inside the aramid fiber (b) and on a silk fiber (c) according to various embodiments;

FIG. 18 is a graph of percent reduction in bacteria after repeated wash and dry cycles of nanocomposite textiles;

FIG. 19A is a facemask and FIGS. 19B-D are SEM images of textiles according to example 6;

FIG. 20 is a bar graph of TGEV (transmissible gastroenteritis virus) particles recovered from treated and untreated nylon-cotton textile specimens in example 6;

FIG. 21 is a bar graph of the log reduction in infectious titer and viral genome copes in nylon-cotton textile in example 6;

FIG. 22 is a bar graph of TGEV (transmissible gastroenteritis virus) particles recovered from treated and untreated face mask textile specimens in example 6; and

FIG. 23 is a bar graph of the log reduction in infectious titer and viral genome copes in face mask textile in example 6; and

FIG. 24 is a bar graph of antibacterial performance for textiles in example 11.

### SUMMARY

Various embodiments include antimicrobial textiles. In some embodiments, the antimicrobial textile may include a sheet substrate comprising a textile and metal oxide nanoparticles in which the nanoparticles are present as a nanocomposite on the surface of and within the sheet substrate such as within the fibers of the textile. The metal oxide included in various antimicrobial textiles may include zinc oxide, for example. The antimicrobial textile may be configured to be worn on a body of a user such as a piece of personal protective equipment like a multilayer face mask in which the antimicrobial textile forms at least one layer of the face mask. In some embodiments, the personal protective equipment may be closing clothing.

The antimicrobial textile may be used for, or included in, various applications. For example, in some embodiments, the antimicrobial textile may also include an adhesive layer, such as when the personal protective equipment comprises a bandage. In some embodiments, the antimicrobial textile may be included in a feminine hygiene product. The antimicrobial textile may be used as a furniture upholstery. In some embodiments, the antimicrobial textile may be a surface cleaning product. In some such embodiments, the surface cleaning product may be a mop, sponge, rag or towel, for example. The antimicrobial textile may also be an article of bedding.

Particular embodiments include antimicrobial face masks including a multilayer sheet portion configured to cover a nose and mouth of a user, one or more of the sheets comprising a metal oxide textile nanocomposite, and straps configured for attachment of the mask to a user's head. In some embodiments, the metal oxide may be zinc oxide.

Other embodiments include methods of making antimicrobial textiles. The method may include applying a metal salt solution to a textile to diffuse the metal salt into the textile, the textile comprising a surface and interior fibers and drying the textile with the applied metal salt solution to bind the metal salt to the surface of and the interior fibers of the textile by forming a nanocomposite of metal nanoparticles or nanostructures in situ. The step of drying the textile may include heating the sheet. The metal salt may include zinc oxide. The resulting antimicrobial textile may then be incorporated into a wearable article, such as an article of personal protective equipment like a face mask worn over the nose and mouth.

### DETAILED SUMMARY OF THE INVENTION

Various embodiments include antimicrobial nanocomposites such as nanocomposite films and method of making the same. The antimicrobial nanocomposites may be antibacterial, antiviral, antifungal, antimold, antimildew, and/or antiparasitic, for example, to kill and/or reduce bacteria, viruses, funguses, mold, mildew and/or parasites. Unless otherwise stated, the use of the term “antimicrobial nanocomposites” as used herein includes, but is not limited to, antimicrobials, antivirals, antifungals, antimolds, antimildews, and/or antiparasitics to kill and/or reduce bacteria, viruses, funguses, mold, mildew and/or parasites. In some embodiments, the antimicrobial nanocomposites may kill, inactivate, and/or reduce the presence of, and/or may reduce the transmission of SARS-CoV-2 (causal agent of COVID-19) or other infectious viruses and bacteria on the surface of or through personal protective equipment. The antimicrobial nanocomposites may be provided as sheet-like layers such as films including functionalized materials like textiles and polymers. Such materials may be used to provide antimicrobial qualities to face masks and other personal protective equipment as well as medical apparel and materials such as bandages. The antimicrobial nanoparticles may be applied to the material, such as through soaking in metal or non-metal ionic salts, and the soaked material may then be dried such as in a commercial dryer to form nanoparticles or nanostructures. In this way, the nanoparticle forms and becomes bound to the material, on the surface and within the material, to form a nanocomposite. This process may be referred to herein as crescoating or crescoating technology. This method may be used to create functionalized materials without the use of environmentally damaging chemicals and without the use of stabilizing or capping agents.

Various antimicrobial nanocomposites as described herein may be used in medical and other patient care, food manufacturing and preparation or close contact environments. The use in medical environments may help reduce the risk of hospital-acquired infection. For example, personal protective equipment that may include antimicrobial nanocomposites include face masks, scrubs, surgical caps, lab coats and shoe covers. Further medical products which may include antimicrobial nanocomposites include patient gowns, sheets, bandages and wound care dressings, and sanitary products. Food production and preparation may include food surfaces.

Other uses for the antimicrobial nanocomposites include bedding such as sheets and blankets. In particular, such bedding may be useful in medical settings such as hospitals and clinics as well as congregate care settings such as assisted living environments. The antimicrobial nanocomposites may be used in apparel outside of the medical setting, such as ordinary consumer apparel like footwear including socks, slipper and shoe lining, undergarments, shirts and pants, as well as industrial apparel such as restaurant or flight attendant uniforms and uniforms for factory workers such as food processing factory workers. The nanocomposites may further be used as upholstery covering furniture and other home good, particularly furniture in high traffic locations such as airports and other transportation hubs, schools, and hotels. In still other examples, the nanocomposites may be used in materials for cleaning surfaces such as rope or sponge heads of mops, sponges, rags and towels.

Various materials may be functionalized using the methods described herein. For example, in some embodiments the material may be porous. It may be a synthetic or a natural textile including synthetic, semi-synthetic, or natural woven or non-woven textiles, fibers, or microfibers. Examples of textiles which may be used include cotton, polyester, nylon, spandex, rayon, linen, cashmere, silk, and wool, acrylic, modacrylic, olefin, acetate, polypropylene, polyvinylchloride, lyocell, latex, aramid, as well as blends or combinations of one or more of these or other materials or fibers. As such, the textile may be natural, such as silk, wool, cotton, cellulosics, flax, jute or bamboo, may be synthetic, such as nylon, polyester, acrylic, spandex, rayon, or a polymer such polypropylene, polyurethane. In some embodiments, the textile may be a mineral such as a glass fiber. Alternatively, the textile may be a blend of different materials including those listed above.

In some embodiments, the material may be a film such as a plastic film, a rigid material such as a rigid plastic, a foam, or a semi-rigid material such as a semi-rigid plastic.

In some embodiments, the material may be a non-woven material such as a material made through a melt blowing process. For example, the material may be a melt-blown polymer such as polypropylene. Such materials may be functionalized with antimicrobial nanoparticles and used as a layer of a multilayer face mask, such as a three-layer face mask, to provide both antimicrobial and filtration effects. In some embodiments, other functionalized materials are used in the face mask as a nanocomposite layer, along with a melt-blown polymer layer as is typically used to trap particulates and which may or may not be functionalized as describe herein, and one or more additional layers such as an inner and/or outer liner. Unless specifically stated otherwise, the term “material” or “textile” as used herein refers generally to all of the materials described herein that may be functionalized, including but not limited to all of the materials stated in the foregoing paragraphs.

Various types of compositions may be used for functionalizing the material. Useful compositions include metals such as transition metals or post-transition metals, metalloids, non-metals, rare earth metals, and alkaline earth metals. The compositions may be in their ionic, elemental and/or nanostructure form, for example. Such nanostructures may be nanoparticles, nanofilms, or other forms.

In some embodiments, the composition is an inorganic nanoparticle made of copper, iodine, silver, tin, zinc, titanium, selenium, nickel, iron, cerium, zirconium, magnesium, manganese, or combinations of more than one of these or other nanoparticles or alloys thereof such as a metal oxide. Examples of metals and metal oxides which may be used in various embodiments include silver, copper oxide, titanium dioxide, and zinc oxide. The metal and metal oxides and other compositions may be used alone or in combination.

In embodiments in which the composition includes nanostructures such as nanoparticles, the nanoparticles may have a size in the nanoscale range, such as between approximately 1 nm and 1000 nm, or between approximately 100 nm and 700 nm, for example. In some embodiments, the nanoparticles may be one or more metal oxides such as titanium dioxide, iron oxide, zinc oxide, copper oxide and silicon dioxide. In other embodiments, the nanoparticles may be non-metals such as selenium.

In some embodiments, the nanoparticles may form a nanocomposite with a porous support material such as sheet-like material. The porous support material may be cotton, cellulose, viscose, silk, aramid, nylon, polypropylene, polystyrene, polyester, polyurethane, polyamide, polyethylene, polycarbonate, or a combination of two of more of these or other materials. The nanocomposite may be a two-phase material including a nanoparticle such as a metal or non-metal nanoparticle on the surface of and within the material such as the fibers throughout the textile or other porous support material.

In some embodiments, the nanocomposite sheet may be used as a product or as a component of a product. In other embodiments, the nanocomposite sheet may be used with an adhesive which may be used to adhere the nanocomposite sheet to a surface of another product or material, either during production of the product or later by a consumer. The nanocomposite sheets may be present as layers such as single, double, triple, or greater numbers of sheets. The nanocomposite sheets may be hydrophobic, hydrophilic, electrostatic, or combinations thereof.

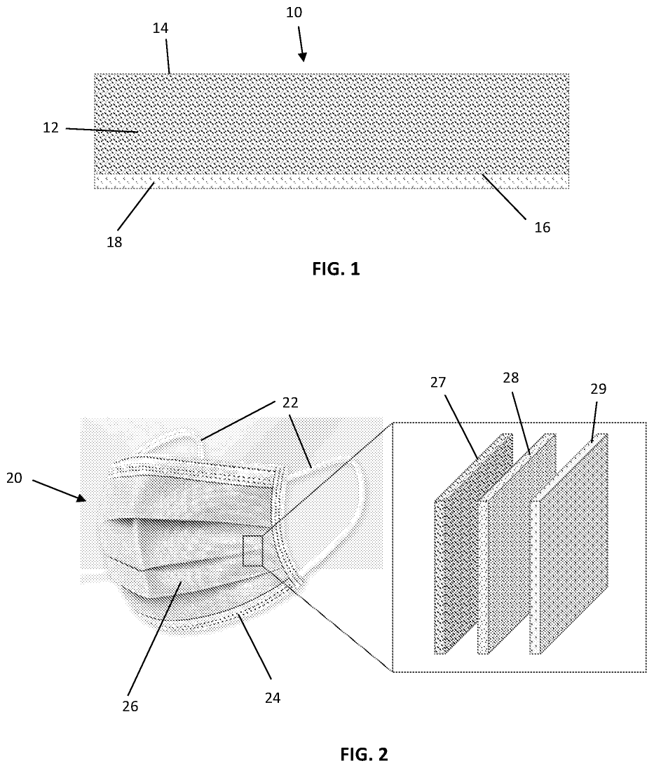

An example of an adhesive nanocomposite sheet is shown in FIG. 1. The sheet 10 includes a nanocomposite sheet 12 having a first surface 14 and an opposing second surface 16. It further includes an adhesive layer 18 adjoined to the second surface 16 of the nanocomposite sheet 12. The adhesive layer 18 may completely cover the second surface 16 as shown or it may be present in a discontinuous manner such as a series of adhesive dots or other patterns. The adhesive layer 18 may be an adhesive such as glue, paste, an electrostatic surface, or any other material that allows reversible or permanent bonding of the sheet 10 to a surface. The sheet 10 may be applied to items which are touched by users, such as touch screens and other interactive surfaces. Depending upon the use, sheet 10 may be transparent such that a user can see through the sheet 10 to the surface of the item. For example, it may be applied to or provided on touch screen of items such as telephones, automatic teller machines, payment portals, etc. It may further be provided on high touch surfaces such as other portions of a cellular phone (the sides and back), handbags, etc.

In other embodiments, the nanocomposite sheet may form one or more layers of personal protective equipment such as face masks. An example of such an embodiment is shown in FIG. 2. In this example, mask 20 includes straps 22 for attachment to the user's head, which may be elastic and may be configured to loop around the user's ear as in this example, or around the user's head as in other configurations, or to tie behind a user's head. The mask 20 may optionally include edging 24 and a filter portion 26 which may be folded as shown or may be smooth. The mask 20 may further include flexible and/or re-shapeable stays in the edging 24, such as a bendable member to shape the mask across the bridge of a user's nose. The filter portion 26 may be a sheet which extends across and covers the nose and mouth of the user and may itself include a plurality of layers including one or more antimicrobial nanocomposite sheets.

The antimicrobial nanocomposite sheets in personal protective equipment may allow the microbes such as bacteria and/or viruses such as COVID-19 or other microbes to be killed and/or inactivated on contact. The antimicrobial nanocomposite sheets may be used in personal protective equipment with or without additional layers such as microbe filtration sheets, or they may additionally function as filtration sheets. By inactivating the microbes on contact, the antimicrobial nanocomposite sheets provide not only a different method of preventing the spread of microbes in the air which may be used as an alternative to or in addition to filtration, but they also reduce the contamination of the surfaces of the personal protective equipment and the risk of spread by touch. Furthermore, because the nanocomposites maintain their antimicrobial effect even after washing, such as after washing 10 times or more, personal protective equipment such as masks made from the antimicrobial nanocomposites have an extended lifespan as compared to traditional filtration materials.

In the example shown in the FIG. 2, the filter portion 26 includes a first layer 27, a second layer 28, and a third layer 29. Both the first layer 27 and the second layer 28 may be antimicrobial nanocomposite sheets which may be hydrophobic. Third layer 29 may be a different material to provide additional user comfort when in contact with a user's face when the mask is in use. For example, third layer may be a liner which may be a soft, hypoallergenic material and may be hydrophobic.

In other examples, the antimicrobial nanocomposite sheets may be used as a component of a feminine hygiene product such as tampons or sanitary pads which absorb menstrual blood. In such embodiments, the antibacterial properties provided by the antibacterial nanocomposite may help to reduce the risk of infection in a user such as toxic shock syndrome due to an overgrowth of group Staphylococcus aureus or toxic shock like syndrome due to group Streptococcus bacteria. For example, the antibacterial nanocomposite may kill, inactivate, prevent, reduce, and/or inhibit the growth of such bacteria in the feminine hygiene product.

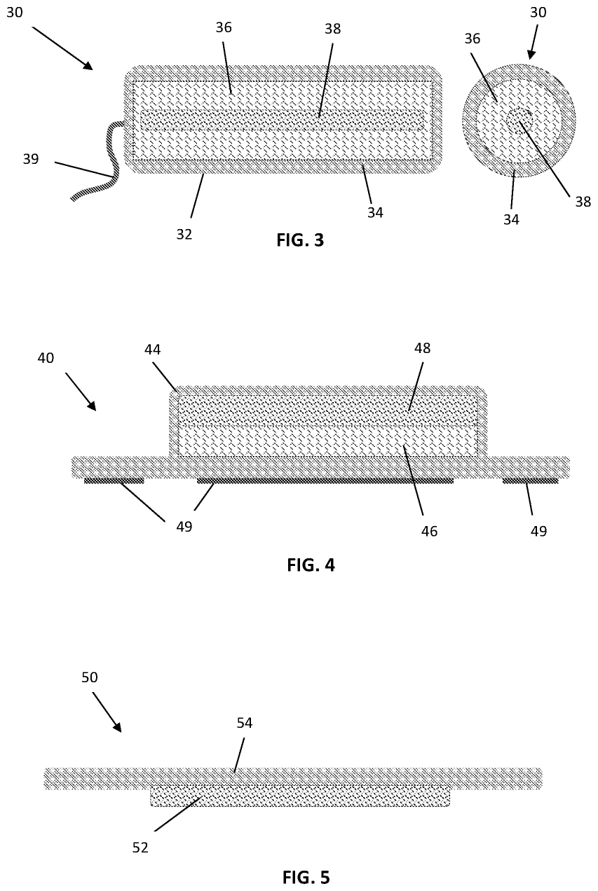

An example of a tampon according to various embodiments is shown in FIG. 3 in which the tampon is shown in longitudinal and axial cross sections. The tampon 30 may include a main body 32 and a string 39 securely attached to one end for removal after use. The main body 32 may include an outer skin contact layer 34, a high absorption layer 36, and an antimicrobial nanocomposite layer 38. Although the antimicrobial nanocomposite layer 38 forms the core of the tampon body 32 in this embodiment, other arrangements and configurations may be used, including multiple high absorption layers 36 and/or multiple antimicrobial nanocomposite layers 38 as well as one or more layers of other materials.

An example of a sanitary pad according to various embodiments is shown in a cross-sectional view in FIG. 4. The sanitary pad 40 includes a skin contact layer 44, a high absorption layer 46, and an antimicrobial nanocomposite layer 48. It further includes adhesive 49 for a user to adhere the sanitary pad 40 to an undergarment. The sanitary pad 40 layers may alternatively include multiple high absorption layers 46 and/or multiple antimicrobial nanocomposite layers 48 which may be in various configurations and may also include additional layers such as a moisture impermeable layer. In alternative embodiments of tampons and sanitary pads, the antimicrobial nanocomposite layer may be constructed of a material which is itself absorbent such that no other absorbent layers are needed, or fewer other absorbent layers are needed.

In still other examples, the antimicrobial nanocomposite sheet may be used in dressings for wounds in order to reduce the risk of infection. Such dressings may be used on general cuts and abrasions, for post-surgical incisions, or in the field for wounds incurred in an accident or during armed conflict, for example. The dressings which include the antimicrobial nanocomposite sheets may be absorbent pads such as gauze pads which may be applied to a wound and held with compression such as by a wrap or may be adhesive bandages, for example. In other embodiments, the antimicrobial nanocomposite sheet may be used in dressings or other materials for cleaning and caring for and maintaining stomas as needed with colostomy bags and dressings, feeding tubes, and ventilator tubes, where the nanocomposite material may reduce the risk of viral or bacterial contamination.

An example of an antimicrobial wound care pad is shown in FIG. 5. The pad 50 includes an adhesive layer 52 (which may be continuous as shown or may be discontinuous) and an antimicrobial nanocomposite layer 54. The pad 50 may further include other layers such as absorbent layers and moisture impermeable layers which may be provided in various configurations. In some embodiments, the antimicrobial nanocomposite layer 54 is constructed of a material which is itself absorbent such that no other absorbent layers are needed.

Various materials such as textile sheets or other sheets or porous materials may be used in the antibacterial nanocomposite sheets, and the antibacterial nanocomposite sheets may be created through a functionalization process. Alternatively, textile threads, fibers or filaments may be functionalized according to the processes described herein, and the functionalized threads, fibers or filaments may subsequently be woven or otherwise formed into textile sheets.

The functionalization process may begin with applying the antimicrobial composition such as the nanoparticle composition to the material to impregnate the material with the composition. The impregnation of the material with the metal salt can be done by immersion or by spraying, for example. In some embodiments the composition is in a suspension or a solution such as an aqueous suspension or solution. While the composition is aqueous in many embodiments, it may alternatively be non-aqueous, such as a dilute solution (such as less than 50% or less than 25%) of an organic solvent such as acetone, ethanol, or isopropanol. Applying the composition to the material may include saturating the support material with the composition, such as by soaking the material in the composition or spraying the composition onto the support material.

Once the composition is applied to the material, the composition may be bound to the material through drying and/or through the application of heat such as through the use of a dryer. The heat may cause evaporation of the solution to initiate thermal reduction and crystallization of the nanoparticles onto the surface of the fibers of the fabric. This may be performed in a large scale through the use of large dryers such as commercial dryers or industrial dryers. Such commercial or industrial dryers may be capable of drying large quantities of material, such as large quantities of textiles, and operating for longer periods, such as continuously throughout the day. For example, such commercial dryers may have larger drying cylinder sizes, higher airflow, and higher BTU ratings which may help to reduce drying time and increase drying efficiency. For example, commercial dryers may have a capacity of 7 cubic feet or greater, or 30 pounds or greater. Industrial dryers may have a capacity of 30 pounds or greater, 50 pounds or greater, or even more. In some embodiments, the dryer may apply heat to the material. In some embodiments, the dryer may blow heated air toward the material and/or move the material while drying such as on a conveyor or by tumbling within the dryer.

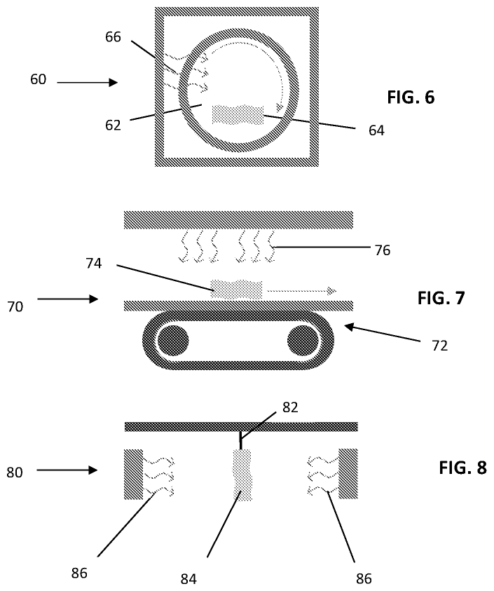

Examples of dryer systems which may be used in various embodiments are shown in FIGS. 6-8. A representation of a dryer 60 is shown in FIG. 6. The dryer 60 includes a dryer chamber 62 and the treated material 64 is inside the dryer chamber 62. The dryer 60 applies heat 66 such as hot air to the material 64 while it is tumbled inside the rotating dryer chamber 62.

Another example of a dryer 70 is shown in FIG. 7. This dryer 70 includes a conveyor system 72. As the treated material 74 passes through the dryer 70 on the conveyor 72, the dryer applies heat 76 such as hot air. The material 74 may pass through the dryer 70 continuously or the conveyor 72 may pause one or more times during passage of the material 74. While the heat is depicted as applied from above, it would alternatively or additionally be applied from any direction to dry the material 74. The source of heat can also an oven, dryer, a heat jet, or a source of infrared light, for example.

In still a further example, the dryer 80 shown in FIG. 8 includes one or more hangers 82 such as hooks or clips for hanging the treated material 84. The soaked material 84 may be hung from a single hook or a plurality of hooks to spread it out to minimize or eliminate folding. The dryer applies heat 86 to the material 84. While heat 86 such as hot air is shown being applied to the material 84 from two opposing sides, it may alternatively or additionally be applied from any or all directions. In some embodiments, the dryer 80 may include a conveyor system to convey the material 84 through and past the heat 86. For example, the hanger 82 may convey the treated material 84 through the dryer 80.

The application of heat to the treated material binds the composition to the material. For example, when nanomaterials are used, they may be grown inside the support material fiber and may be held physical in place by the surrounding material. The use of energy such as heat may facilitate crystallization.

Following heat treatment, the composition may remain bound to the material for an extended period of time. For example, the composition may remain bound to the material during one or more subsequent uses and/or washes such as laundry cycles.

In some embodiments, the composition may be added to a textile material during a laundry process. For example, the composition may be added to textile while the textile is being washed, such as during the wash cycle of a laundry machine. The composition may be provided as an additive to the laundry washing detergent or may be separately added during the washing cycle. The additive may be an aqueous solution of a metal or non-metal salt, for example. When the wash cycle is complete, the textile may be dried in a laundry dryer according to the normal laundry process. In this way, the textile may be functionalized during routine laundry processes. This may be particularly useful for industrial purposes, such as hotels, hospitals, nursing homes, and other facilities, to provide antimicrobial qualities to materials such as bedding (sheets, blankets, pillowcases, pillows, mattress covers) and/or clothing such as scrubs worn by medical personnel or gowns or other clothing worn by patients

### WORKING EXAMPLES

### Example 1

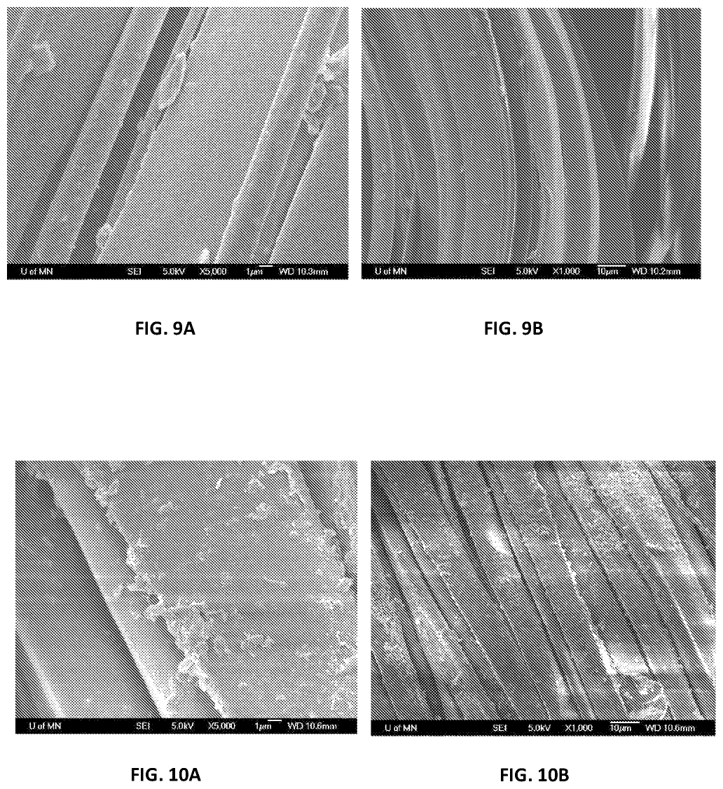

A nanocomposite textile was prepared by soaking a textile in a zinc salt solution. The textile was a blend of cotton, polyester, nylon and spandex. The textile was then dried in a commercial dryer at a temperature of around 65 degrees Celsius. After the drying was complete, the zinc oxide nanocomposite textile was washed and examined under scanning electron microscopy (SEM). For comparison, FIGS. 9A and 9B show SEM photographs of the untreated textile at 1000× and at 5000×, respectively. FIGS. 10A and 10B show SEM photographs of the same textile after treatment to form a zinc oxide nanocomposite at 1000× and 5000×. The comparisons show that substantial coating of the textile occurred, which was maintained even after washing.

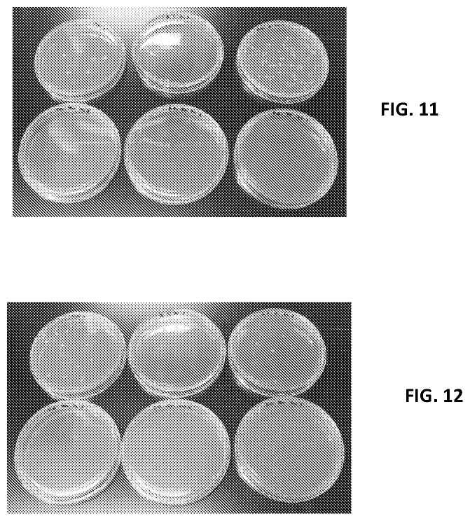

The zinc oxide nanocomposite textile prepared as described above was tested for antimicrobial properties using American Association of Textile Chemists and Colorists (AATCC) Test Method 100-2004. The experiment was repeated in triplicate, using both treated and untreated textile. Two bacterial species were used for testing: Pseudomonas aeruginosa, a Gram-negative bacterium, and Staphylococcus aureus, a Gram-positive bacterium. In each case, the textile was inoculated with the bacteria in broth, while a control was treated in the same manner but with broth alone. The textiles were allowed to incubate for 24 hours. The microbial concentrations on the textile were then determined by elution of the textile in neutralizing broth, dilution, and plating on Petri dishes. The resulting bacteria growth on the Petri dishes is shown in FIGS. 11 and 12.

FIG. 11 is a photograph of the Petri dishes resulting from the test with P. aeruginosa, while FIG. 12 is a photograph of the Petri dishes resulting from the test with S. aureus. In each case, Petri dishes on the upper row are the results for untreated textile with the center Petri dish as the control (no bacteria), while the Petri dishes in the lower row are the results for treated textile. For both bacteria, the bottom row of Petri dishes for the treated textiles had no bacterial growth, while the upper row of Petri dishes for the untreated textile had numerous bacterial colonies. The zinc oxide nanocomposite textile exhibited complete bacterial control. These results show that the process of preparing a textile as described herein was effective for functionalizing the textile for antibacterial properties that persisted even after a washing.

### Example 2

Polyester, aramid, wool, silk, and nylon/cotton were functionalized with ZnO nanoparticles. The textiles were functionalized by soaking in an aqueous solution of zinc salts including zinc nitrate, zinc acetate, zinc sulfate, and zinc chloride at an ideal concentration range of 0.1 to 0.75M for 30 minutes followed by heating in a conventional oven at 100 degrees Celsius or a dryer at 60 degrees Celsius until dry. The resulting functionalized textiles had nanoparticle loading of 1-3% w/w. The nanocomposite functionalized textiles were then tested for antibacterial activity in triplicate according to AATCC Test Method 100-2004 using Pseudomonas aeruginosa (PA) (Gram negative) and Staphylococcus aureus (SA) (Gram positive) as described above in Example 1. The antimicrobial properties of the zinc nanocomposite textiles were tested before wash and after undergoing 1, 5, and 10 wash cycles.

Before-wash antibacterial test: According to the AATCC protocol, the samples were treated at 0 hours (immediate elution), and then left for 24 hours. The harvested bacteria were plated on petri dishes to calculate the reduction in bacterial growth. The results are reported in % reduction and calculated by Equation 1:

R=100(B−A)/B (Eq. 1),

where R is the % reduction, A and B are the number of bacteria obtained from the inoculated treated test specimen swatches in the jar recovered either after incubation over the desired contact period “A”, or immediately after inoculation at “0” contact time) “B”. The same formula was used for 24 hours elution.

For all experiments, Equation 2 was used to evaluate if the bacterial source is effective, meaning if the initial concentration of bacteria used was enough to perform antimicrobial testing. Equation 2:

E=Log(B)−Log(A) (Eq. 2),

where E is the effective concentration, A and B are the number of bacteria recovered from inoculated untreated control textile immediately after inoculation, and after 24-hour incubation, respectively. E must be greater than 1.5 to be deemed effective.

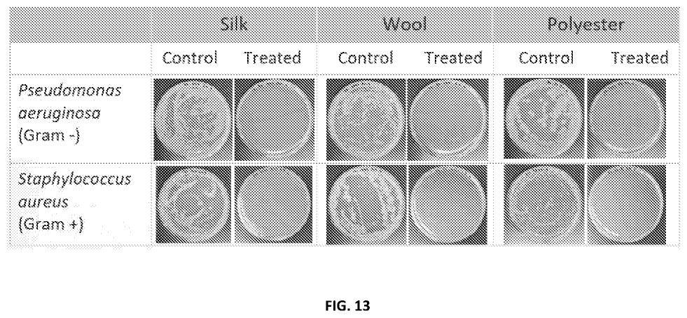

In all experiments, the value of Log(B)−Log(A) ranged from 3 to 5, which confirms the effective bacterial concentration. Images of some of the resulting petri dish plates for the antimicrobial tests are shown in FIG. 13. The results show excellent antimicrobial properties for the nanocomposite textiles as compared to untreated textiles.

Table 1 shows the results of the antimicrobial tests for the percent reduction of bacteria at both 0 hours (immediate elution) and after 24 hours of incubation with ZnO nanocomposite textiles. The immediate elution data reveals a reduction between 55% and 75% for the different textiles, which is an unexpected positive result since the bacteria were only exposed to the textiles for a few seconds. The data shows some variable effect on Pseudomonas aeruginosa (PA) and Staphylococcus aureus (SA). For some samples, negative values were observed and indicate that there were more bacteria recovered than the control. This could be due to variable adsorption properties of the textiles or to improper preparation of the bacterial concentrations. For longer elution, Table 1 shows that the nanocomposite textiles killed 100% of the bacteria that were exposed to them for 24 hours.

TABLE 1

| | % reduction after 0 hours | | % reduction after 24 hours | |
|---|---|---|---|---|
| Sample | PA | SA | PA | SA |
| Wool | 62.64 | 55.50 | 99.2 | 100 |
| Silk | 74.66 | NA | 100 | 100 |
| Polyester | NA* | NA | 100 | 69.23 |
| Aramid | NA | 70.37037 | 66.6 | 100 |
| Nylon/Cotton | 66 | NA | 100 | 100 |

*“NA” is used here for plate contamination.

The zinc nanocomposite textiles were washed 1, 5 and 10 times using AATTC approved washing and drying machines to assess the durability of the antimicrobial textiles. Antibacterial properties were then tested according to the same protocol and using the same formula described above. Table 2 summarizes the effects of wash cycles on the antimicrobial properties after immediate elution (0 hours). Table 2 shows the effect of wash cycles on bacterial reduction (%) of Pseudomonas aeruginosa (PA) and Staphylococcus aureus (SA) after 0 hours of incubation with zinc nanocomposite textiles. The data shows that the nanocomposite textiles retained their excellent antimicrobial properties for at least 10 wash cycles, indicating the high stability of the nanocomposite textiles.

TABLE 2

| | % reduction after immediate elution (0 hour incubation) | | | | | |
|---|---|---|---|---|---|---|
| | PA | SA | PA | SA | PA | SA |
| | Wash cycles | | | | | |
| Sample | 1 cycle | | 5 cycles | | 10 cycles | |
| Wool | 98.57 | 91.09 | 93.39 | 93.06 | 94.45 | 95.96 |
| Silk | 100.00 | 95.20 | 100 | 100.00 | 100.00 | 97.26 |
| Polyester | NA* | 95.00 | 74.59 | 100.00 | 98.92 | 100.00 |
| Aramid | NA | 33.33 | NA | NA | NA | NA |
| Nylon/Cotton | 93.31 | 33.33 | NA | 95.63 | 99.62 | 96.88 |

*“NA” is used for either negative values or when there is plate contamination.

### Example 3

The experiment of Example 2 was repeated but with the initial salt concentration during synthesis of the nanocomposite textile increased to double the nanoparticle loading of the textile, specifically zinc nitrate, zinc acetate, zinc sulfate, and zinc chloride at an ideal concentration range of 0.75 to 1.5M. The resulting textiles had a nanoparticle loading of 3-6% w/w. The results were compared to those of Example 2 to evaluate the effect of concentration of nanoparticles on the antimicrobial properties of textiles. Table 3, below, shows the before-wash test results for the textiles of this example in comparison to the before wash test results for the textiles of Example 2. Table 3 shows the effect of nanoparticles loading on antimicrobial properties for Pseudomonas aeruginosa (PA) and Staphylococcus aureus (SA) after immediate elution (0-hour incubation). These results show that the textiles can exhibit 100% antimicrobial efficiency, even at immediate elution (0 hours contact time), by increasing the initial salt concentration during the synthesis process to obtain a final nanoparticle loading of 3-6% in the nanocomposite textile. This is a remarkable performance that can be extremely useful to rapidly inactivate bacteria or viruses within seconds of contact with the textiles, opening new avenues for applications in personal protective equipment (PPE) such as medical masks, gowns, scrubs, and lab coats.

TABLE 3

| | % reduction after immediate elution (0 hours) | | | |
|---|---|---|---|---|
| | Nanoparticle loading: | | Nanoparticle loading: | |
| | 1-3% w/w | | 3-6% w/w | |
| Sample | PA | SA | PA | SA |
| Wool | 62.64 | 55.50 | 98.57 | 91.09 |
| Silk | 74.66 | NA | 100.00 | 95.20 |
| Polyester | NA* | NA | NA | 95.00 |
| Aramid | NA | 70.37037 | NA | 33.33 |
| Nylon/Cotton | 66 | NA | 93.31 | 33.33 |

*“NA” is used for either negative values or when there is plate contamination.

As in Example2, the nanocomposite textiles of this experiment were washed for 1, 5, and 10 wash cycles to test the effect of washing on the antimicrobial properties of the nanocomposite textiles using the same pathogens. The results are shown below in table 4, in which the effect of wash cycles on percent bacterial reduction of Pseudomonas aeruginosa (PA) and Staphylococcus aureus (SA) after 24 hours incubation with zinc nanocomposite textiles is shown for 1, 5 and 10 wash cycles. The results demonstrate that the antimicrobial properties were retained even after 10 wash cycles for most of the textiles. For 10 wash cycles, the nanocomposite textiles showed more than 90% bacterial reduction for Staphylococcus aureus. The antimicrobial activity decreased by 20% and 40% for wool and silk respectively for Pseudomonas aeruginosa. We previously observed a similar behavior on selenium/polyurethane nanocomposite. This could be explained by the fact that Gram-negative bacteria such as Pseudomonas aeruginosa are usually more resistant to antimicrobial agents because of their extra polysaccharide layer outside the cell wall. Thus, for some textiles, such as wool, silk and selenium/polyurethane, the nanocoating functionalization may be repeated, such as through the processes described herein, after the textile is washed several times such as 10 times or more. This may be appropriate for use against Gram-negative bacteria such as Pseudomonas aeruginosa.

TABLE 4
% reduction after 24 hours incubation

| | Wash cycles | | | | | |
|---|---|---|---|---|---|---|
| | 1 cycle | | 5 cycles | | 10 cycles | |
| | Microorganism | | | | | |
| Sample | PA | SA | PA | SA | PA | SA |
| Wool | 100 | 100 | 96.6 | 100 | 60 | 100 |
| Silk | 100 | 100 | 99.5 | 100 | 89 | 83 |
| Polyester | 16.8 | 100 | NA | 100 | NA | 72.5 |
| Aramid | NA** | NA | NA | NA | NA | NA |
| Nylon/Cotton | 60.70 | 100 | NA | 98 | NA | 100 |

*“NA” is used for either negative values or when there is plate contamination.

### Example 4

Samples of the nanocomposite textiles produced as described herein were characterized by the University of Minnesota Characterization Facility.

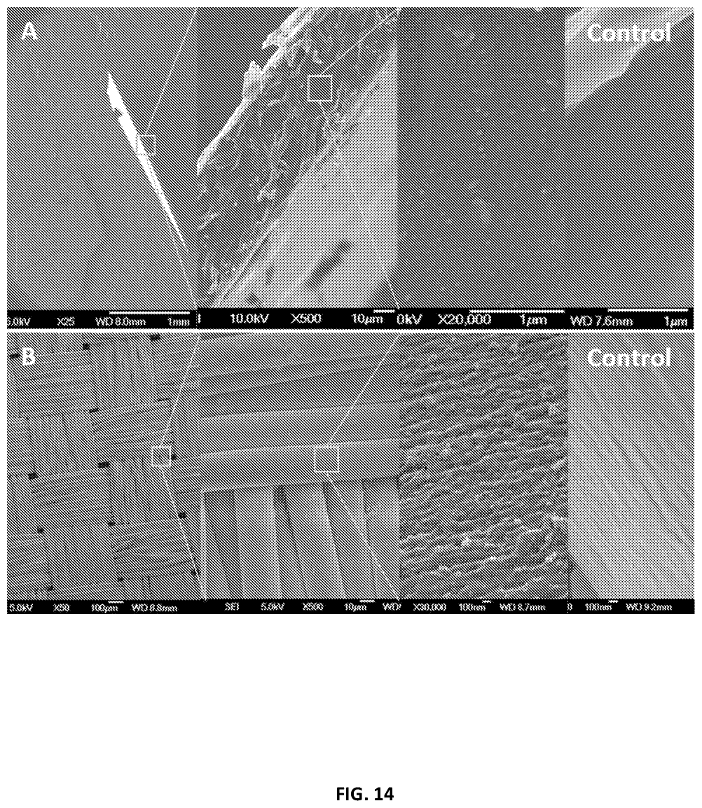

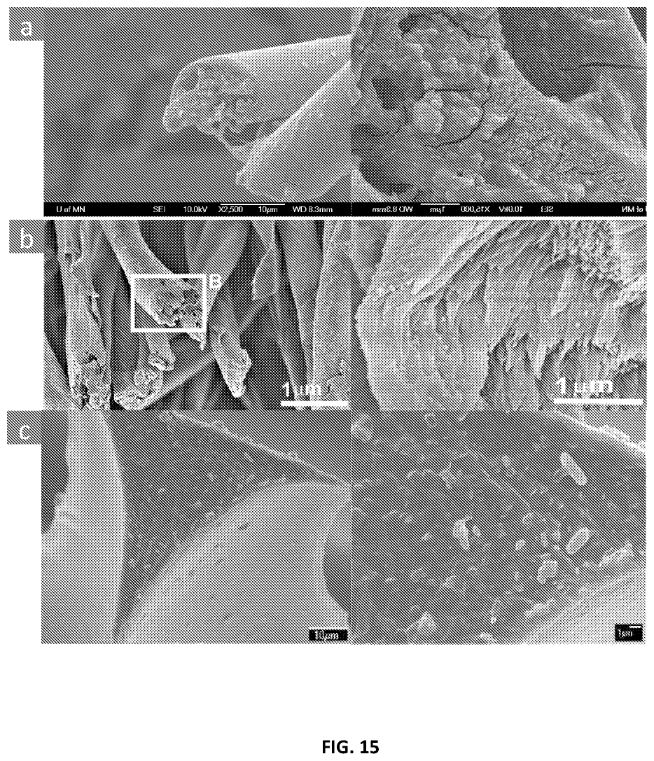

Morphological analysis of the nanocomposite textiles was performed including analysis of the structural integrity of the fibers, the size, shape, and homogeneity of distribution of nanoparticles, and the interface between the nanoparticles and the fibers. Assessment of these characteristics was performed using scanning electron microscopy (SEM). FIGS. 14 and 15 shows some examples of the SEM images obtained for nanocomposite materials produced as described herein.

In FIG. 14, row A shows SEM images of zinc-polyurethane nanocomposite film at 25×, 500× and 20,000× verses a control of the same polyurethane without a nanocomposite at 20,000×. The arrows show two pieces of the nanocomposite thin film. Image amplification at the film cross-section shows the presence of zinc nanoparticles inside the film. The SEM images in FIG. 14 row B are zinc-nylon nanocomposite at 50×, 500× and 30,000× verses a control of the same nylon at 30,000×. The zinc nanoparticles can be seen embedded within the nylon fibers.

In FIG. 15, the SEM images of cross-sections of textile fibers show the formation of nanoparticles inside the bulk of the fibers. In FIG. 15, row A are SEM images of a zinc-aramid nanocomposite at 2,500× and 15,000×. Row B of FIG. 15 are SEM images of a silver-polyester/cotton nanocomposite at 1200× and 25,000×, and row C of FIG. 15 are SEM images of an iron-polyurethane nanocomposite at 1200× and 4500×.

The chemical and crystalline structures of the nanoparticles were also evaluated. The crystalline phase of the nanoparticles has a significant influence on their functionality. Nanoparticles were recovered from the cotton/polyester textiles by grinding. The powder was analyzed using Energy Dispersive Spectroscopy (EDS) and X-ray diffraction (XRD). Zinc oxide, nano titanium (TiO₂) and nanoceramics were analyzed for subsequent functionality testing.

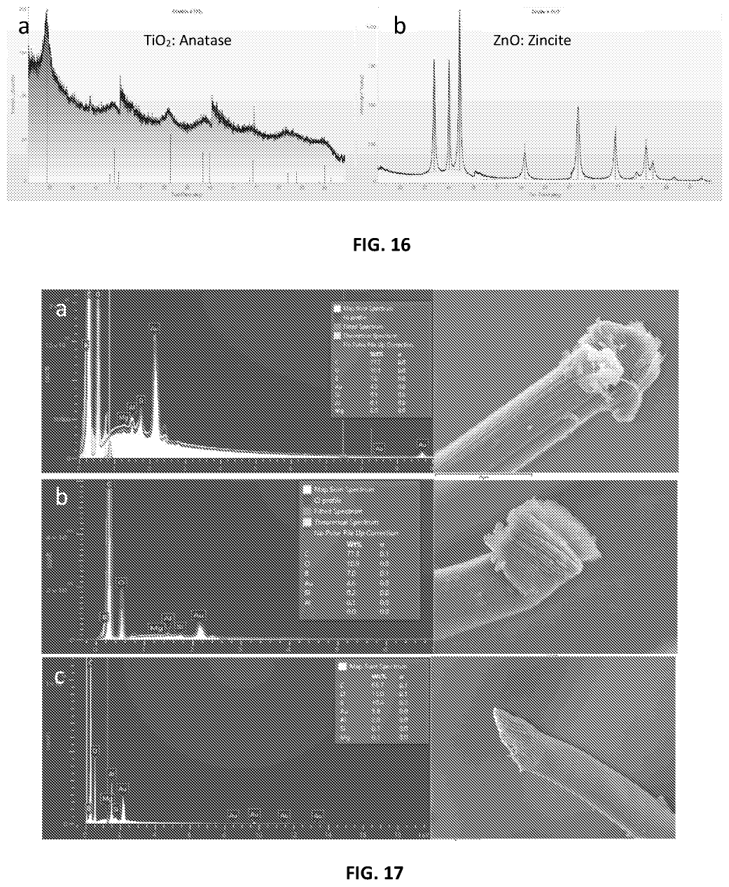

The XRD results are shown in FIG. 16, which shows the x-ray diffraction spectra of TiO₂ nanoparticles (a) and ZnO nanoparticles (b). These results confirmed the presence of ZnO and TiO₂ nanoparticles and revealed that ZnO nanoparticles were mostly present in a crystalline phase called Zincite, while TiO₂ nanoparticles were present in a crystalline phase named Anatase.

The characterization of the ceramic nanoparticles on aramid and silk were conducted using SEM and EDS and these results are shown in FIG. 17. FIG. 17 shows the EDS-SEM data for ceramic nanoparticles on the surface of an aramid fiber (a), inside the aramid fiber (b), and on a silk fiber. The EDS showed that the ceramic nanoparticles are composed of boron, silicon, and aluminum, and they are present both on the surface and in the bulk material of the fibers.

### Example 5

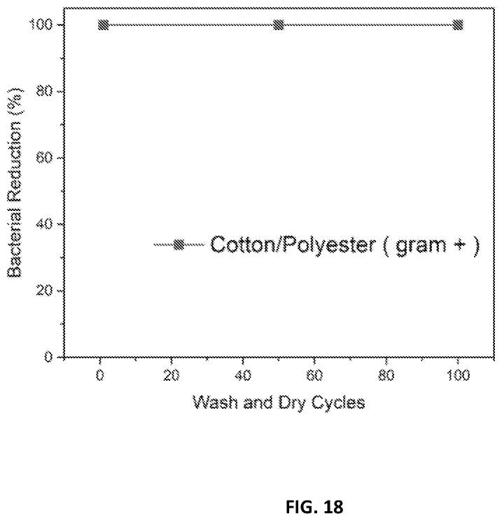

A nanocomposite textile was prepared by soaking a textile in an aqueous solution of zinc nitrate, zinc acetate, zinc sulfate, and zinc chloride at an ideal concentration range of 0.1 to 0.75M. The textile was a cotton polyester blend NIKE sock. The textile was then dried in a commercial dryer at a temperature of around 60 degrees Celsius. The textile was submitted to 100 wash and dry cycles. The zinc oxide nanocomposite textile prepared as described above was tested for antimicrobial properties using AATCC Test Method 100-2004 before the wash and dry cycles, after 50 wash and dry cycles, and at the end of the 100 wash and dry cycles. Staphylococcus aureus, a Gram-positive bacterium, was used for testing. The textile was inoculated with the bacteria in broth, while a control was treated in the same manner but with broth alone. The textiles were allowed to incubate for 24 hours. The microbial concentrations on the textile were then determined by elution of the textile in neutralizing broth, dilution, and plating on Petri dishes. Untreated control socks were tested for comparison of the antimicrobial effect of the nanocomposite socks. The results are shown in the graph presented in FIG. 18. The antimicrobial properties of the nanocomposite textiles before washing and drying, after 50 wash and dry cycles, and after 100 wash and dry cycles were stable.

### Example 6

Textile samples prepared according to the invention described were tested to evaluate the virucidal efficacy of the textiles against a surrogate to SARS-CoV-2 [(transmissible gastroenteritis virus (TGEV)] as described in Gonzalez, Andrew, et al. “Durable Nanocomposite Face Masks with High Particulate Filtration and Rapid Inactivation of Coronaviruses.” (2021). DOI: 10.21203/rs.3.rs-821052/v1

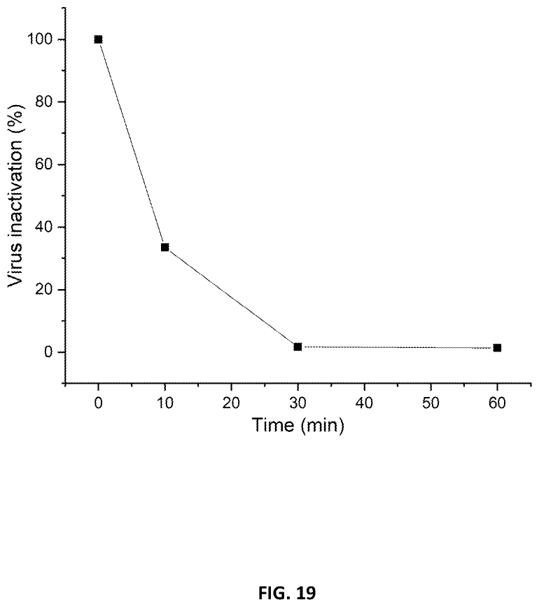

Two types of textiles were used in this example, a nylon-cotton textile and a melt-blown polypropylene material of the type used in face masks. The textiles used in this example were prepared by soaking the textile in zinc nitrate, zinc acetate, zinc sulfate, and zinc chloride at an ideal concentration range of 0.1 to 0.75M and drying in a commercial oven at 100 degrees Celsius to evaporate the water and initiate nucleation and growth of the zinc oxide nanoparticles. The resulting nanoparticles or nanostructures were randomly distributed within and on the surface of the material and varied in shape and size from 5-500 nm. The SEM images presented in FIG. 19 show the growth of nanoparticles not only on the surface but also within the bulk of the material. FIG. 19A shows an example of a facemask. FIG. 19B is an SEM image of the untreated polypropylene textile, while FIG. 19C is an SEM image of the zinc-polypropylene nanocomposite textile with “petal” shaped zinc particles. FIG. 19D shows SEM images of the polyester-cotton textile after treatment, at various levels of magnification. The images show internal nanoparticle growth. Mass measurements before and after growth revealed a nanoparticle loading of 3-6% by mass of the final composite.

After drying, the face mask was thoroughly washed in a commercial washing/drying unit according to the standard AATCC LP1: Machine Wash protocol. In the tests, 60 treated samples were compared to 60 untreated control samples for each fabric.

The TGEV (transmissible gastroenteritis virus), an alpha coronavirus causing gastrointestinal infections in pigs, was used as a surrogate to SARS-CoV-2. The TGEV was propagated and titrated in ST (swine testicular) cells. The cells were grown in Eagle's MEM medium supplemented with antibiotics and fetal bovine serum.

Aliquots (1 mL) of the virus recovery medium (MEM medium with 4% FBS) were distributed in 27 round bottom 13 mL plastic centrifuge tubes (Falcon). The 27 virus recovery tubes were divided into 3 groups of 9 tubes each. Group 1 was marked as control, group 2 marked as treatment, and group 3 was marked as leached particles control (treated control). In each group, 3 tubes were assigned for virus recovery at 3 different time points (10 min, 30 min, and 60 min).

Two square Petri dishes were marked as control and treatment. Parafilm squares (2×2 cm²) were cut and 9 parafilm squares were placed in each Petri dish. 4-Aliquots (75 μL) of TGEV suspension (with initial titer=˜6.5 Log TCID₅₀/mL) were placed on the center of each parafilm square. Nine untreated (control) and 9 treated nylon/cotton specimens were placed over the surface of each parafilm square in the control-marked and treatment-marked Petri dishes, respectively, where the virus droplets were sandwiched between the tested textile and the parafilm squares. The virus droplets were absorbed immediately by the textile specimens as they are hydrophilic.

After each contact time point (10 min, 30 min, and 60 min), a set of 3 samples (in triplicate) was withdrawn from the control and treatment Petri dishes and each sample set (tested specimen with the absorbed virus and the parafilm square) was transferred into its corresponding virus recovery tube of group 1 and 2. To recover the surviving viral particles, all virus recovery tubes were vortexed for 2 min immediately after transferring the sample set in each of them.

In virus recovery group 3, a treated textile specimen was transferred first in each tube and vortexed for 2 min before adding 75 μL aliquot of the virus directly into each tube (without direct contact with the fabric). This was done to know whether a fraction of viral particles was inactivated by contact with the textile active ingredients that were possibly leached in the virus recovery solution following the recovery of the virus from the fabric.

The titer of surviving virus recovered in the recovery medium was performed by the 50% tissue culture infective dose (TCID₅₀) method. Serial 10-fold dilutions were prepared from the recovery medium of each sample. These dilutions were inoculated in 80% confluent monolayers of ST cells, prepared in 96-well microtiter plates using 3 wells per dilution (100 μL of each sample dilution/well).

The infected cells were incubated at 37° C. in a 5% CO² -incubator for up to five days and examined daily under an inverted microscope for the appearance of cytopathic effects (CPE). The highest dilution of the virus, which produced CPE in 50% of the infected cells, was considered as the endpoint. The titer of the surviving virus in each sample was then calculated by the Karber method (Karber, G. (1931). 50% end point calculation. Archivfitr Everithentelle Pathologic and Pharmakologie, 162, 480-483) and expressed as log₁₀ TCID₅₀/sample.

The entire experiment was repeated one more time on a separate day. Both experiments used triplicate samples for each contact time and hence the results are shown as an average of 6 replicates.

To gain some insight on the mode of action on virus inactivation, we quantified the viral genome copy numbers in the elution buffer after virus recovery from the control and treated samples. Viral RNA was extracted from 140 μL of sample using QIAamp DSP Viral RNA Mini Kit (Qiagen, Germany) according to the manufacturer's instructions. The RNA was eluted in 100 μL of elution buffer and stored at −80° C. until used for viral genome quantification. For RT-qPCR, we used PCR primer set and probe shown in Table 5. The RT-qPCR primers were designed to target a conserved 146 bp region [corresponding to the region between nucleotides 370 and 515 of the TGEV S gene with reference to (with reference to the sequence of TGEV- GenBank accession no.: KX900410.1)]. The primers and probe were manufactured by Integrated DNA Technologies (IDT, Coralville, IA). The reactions were performed using AgPath-ID One-Step RT-PCR kit (Applied Biosystems by Thermo Fisher Scientific, Waltham, MA).

The reaction mixture (25 μL) consisted of 5 μL of template RNA, 12.5 μL of 2× RT-PCR buffer, 1 μL 25× RT-PCR Enzyme Mix, 0.50 μL of 10 μM forward primer (200 nM final concentration), 0.50 μL of 10 μM reverse primer solution (200 nM final concentration), 0.30 μL of 10 μM probe (120 nM final concentration), and 5.20 μL of nuclease-free water. The RT-qPCR was performed in the QuantStudio™ 5 PCR thermocycler system (Thermo Fisher Scientific, Applied Biosystems™, catalog number: A28140). Reverse transcription was performed at 45° C. for 10 min. Taq polymerase activation was done at 95° C. for 15 min followed by 45 amplification cycles using a 95° C./15 s denaturation step and an annealing/extension step at 58° C. for 45 s. Fluorescence was measured at the end of annealing step in each cycle. In each run of RT-qPCR, standard curve samples and no template control were used as positive and negative controls, respectively.

The TGEV standard/calibration curve was constructed for absolute quantification of viral genome copy number, in which we used serial ten-fold dilutions of a 557 bp RT-PCR purified amplicon of TGEV S gene (including the 146 bp target sequence of the RT-qPCR prime/probe set). The 557 bp TGEV S gene fragment was produced by RT-PCR reaction using an in-house developed primer set shown in Table 5. Results were expressed as cycle threshold (Ct) values. The Ct values and standard curve were used to calculate the absolute genome copy number of TGEV, expressed as genome copies/sample.

Table 5 below shows the oligonucleotides for TaqMan-based TGEV RT-qPCR used for each PCR reaction in this example. A + polarity indicates virus sense and a − polarity indicates anti-virus sense. The position is the corresponding nucleotide position of TGEV genome (GenBank accession no.: KX900410.1) as reference.

TABLE 5

| | Oligo- | | | | Product | |
|---|---|---|---|---|---|---|
| PCR | nucleotide | | Polar- | Pos- | length | |
| rxn | name | Sequence (5′→3′) | ity | ition | (pb) | Ref. |
| TGEV RT- | TGEV-F | TCTGCTGAAGGTGCTATTA | + | 20722- | 146 bp | [1] |
| qPCR | | TATGC | | 20745 | | |
| | TGEV-R | CCACAATTTGCCTCTGAAT | − | 20867- | | |
| | | TAGAAG | | 20843 | | |
| | TGEV-P | FAM- | + | 20751- | | |
| | | TAAGGGCTC/ZEN/ACCACC | | 20776 | | |
| | | TACTACCACCA-3IABKFQ | | | | |
| TGEV RT- | TP-F | GCAGGTTACCACCTAATTC | + | 20486- | 557 bp | Pre- |
| PCR | | AGA | | 20507 | | pard |
| (For standard | TP-R | CAGGATTAAACCACCAAA | − | 21043- | | in- |
| curve con- | | GGTC | | 21022 | | house |
| struction) | | | | | | |

[1]Vemulapalli, R., Gulani, J., & Santrich, C. (2009). A real-time TaqMan ® RT-PCR assay with an internal amplification control for rapid detection of transmissible gastroenteritis virus in swine fecal samples. Journal of virological methods, 162(1-2), 231-235.

The results presented here are the geometric means of 6 replicates. One-way ANOVA was performed and the significance of differences between the means were performed by paired comparison using Tukey test at significance=0.05. Table 6 below is a summary of the surviving TGEV titers and number of TGEV genome copies recovered from Nylon/cotton textile specimens after 10, 30, and 60 min contact times.

TABLE 6

| | | | | | | | |
|---|---|---|---|---|---|---|---|
| Exposure | | | | | | | |
| time | Date of | | Untreated | Treated | Treatment- | Log | % |
| (min) | testing | Replicate | sample | sample | control | reduction | reduction |

Log₁₀ TCID₅₀/sample

| | | | | | | | |
|---|---|---|---|---|---|---|---|
| 10 | Oct. 5, 2020 | R1 | 5.17 | 2.17 | 5.50 | 3.00 | 99.90000 |
| | | R2 | 5.50 | 2.50 | 5.83 | 3.00 | 99.90000 |
| | | R3 | 5.17 | 2.50 | 5.50 | 2.67 | 99.78620 |
| | Oct. 12, 2020 | R4 | 5.17 | 1.50 | 5.83 | 3.67 | 99.97862 |
| | | R5 | 5.83 | 1.50 | 5.17 | 4.33 | 99.99532 |
| | | R6 | 5.83 | 1.50 | 5.17 | 4.33 | 99.99532 |
| | Oct. 5, 2020 | R1 | 5.17 | 2.17 | 5.83 | 3.00 | 99.90000 |
| | | R2 | 5.50 | 1.83 | 5.50 | 3.67 | 99.97862 |
| | | R3 | 5.50 | 1.83 | 5.50 | 3.67 | 99.97862 |
| 30 | Oct. 12, 2020 | R4 | 5.83 | 1.50 | 5.50 | 4.33 | 99.99532 |
| | | R5 | 5.83 | 1.50 | 5.17 | 4.33 | 99.99532 |
| | | R6 | 5.50 | 1.50 | 5.83 | 4.00 | 99.99000 |
| 60 | Oct. 5, 2020 | R1 | 5.50 | 1.50 | 5.50 | 4.00 | 99.99000 |
| | | R2 | 5.17 | 1.50 | 5.17 | 3.67 | 99.97862 |
| | | R3 | 5.17 | 1.83 | 5.50 | 3.34 | 99.95429 |
| | Oct. 12, 2020 | R4 | 5.17 | 1.50 | 5.50 | 3.67 | 99.97862 |
| | | R5 | 5.83 | 1.50 | 5.17 | 4.33 | 99.99532 |
| | | R6 | 5.83 | 1.50 | 5.50 | 4.33 | 99.99532 |

Log₁₀ viral genome copy number/sample

| | | | | | | | |
|---|---|---|---|---|---|---|---|
| 10 | Oct. 12, 2020 | R1 | 8.04 | 6.96 | 8.21 | 1.09 | 91.79958 |
| | | R2 | 8.14 | 6.71 | 8.16 | 1.44 | 96.35588 |
| 30 | | R1 | 8.14 | 6.54 | 8.25 | 1.60 | 97.50288 |
| | | R2 | 8.08 | 6.54 | 8.29 | 1.54 | 97.09515 |
| 60 | | R1 | 7.99 | 6.59 | 8.16 | 1.40 | 96.00742 |
| | | R2 | 8.01 | 6.51 | 8.16 | 1.50 | 96.84208 |

TCID₅₀ = 50% Tissue Culture Infectivity Dose

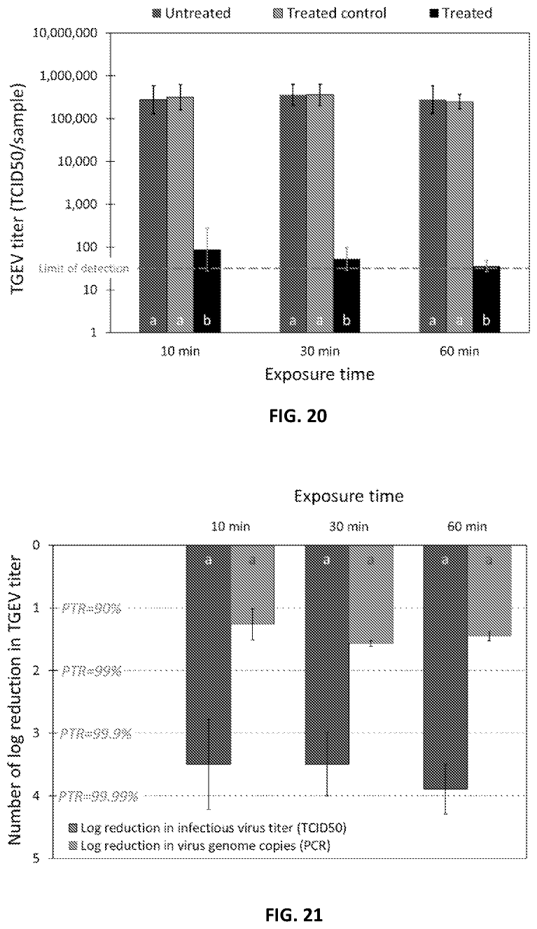

The titer of infectious TGEV particles recovered from nylon-cotton textile specimen after 10, 30, and 60 min contact times are shown in the bar graph presented as FIG. 20. The columns are the geometric mean of 6 replicates. The error bars represent the ±one geometric standard deviation. The scattered green line is the limit of detection. Same letters at each column base indicate geometric means that are not significantly different from one another at each contact time p≥0.05. At each time point, the first bar is untreated, the second bar is the treated control, and the third bar is the treated textile samples.

The number of log reduction in the infectious titer (each first bar) and viral genome copies (each second bar) after 10, 30, and 60 min contact times with Nylon-cotton textile specimens are shown in the bar graph presented as FIG. 21. The columns are the arithmetic mean and the error bars represent ±one standard deviation. Same letters at each column base indicate geometric means that are not significantly different from one another at each contact time p≥0.05. PTR=percentage of virus titer reduction.

A summary of the surviving TGEV titers and number of TGEV genome copies recovered from facemask textile specimens after 10, 30, and 60 min contact times are shown below in Table 7. TCID₅₀ is the 50% Tissue Culture Infectivity Dose.

TABLE 7

| | | | | | | | |
|---|---|---|---|---|---|---|---|
| Exposure | | | | | | | |
| time | Date of | | Untreated | Treated | Treatment- | Log | % of titer |
| (min) | testing | Replicate | sample | sample | control | reduction | reduction |

Log10 TCID₅₀/sample

| | | | | | | | |
|---|---|---|---|---|---|---|---|
| 10 | Oct. 5, 2020 | R1 | 4.50 | 2.17 | 4.17 | 2.33 | 99.53226 |
| | | R2 | 4.50 | 1.50 | 4.50 | 3.00 | 99.90000 |
| | | R3 | 4.50 | 1.5 | 4.50 | 3.00 | 99.90000 |
| | Oct. 12, 2020 | R4 | 5.50 | 2.17 | 5.83 | 3.33 | 99.95323 |
| | | R5 | 5.83 | 1.50 | 6.17 | 4.33 | 99.99532 |
| | | R6 | 5.83 | 2.17 | 5.83 | 3.66 | 99.97812 |
| 30 | Oct. 5, 2020 | R1 | 4.17 | 1.5 | 4.50 | 2.67 | 99.78620 |
| | | R2 | 4.83 | 1.5 | 4.17 | 3.33 | 99.95323 |
| | | R3 | 4.50 | 1.5 | 3.50 | 3.00 | 99.90000 |
| | Oct. 12, 2020 | R4 | 5.83 | 1.5 | 5.83 | 4.33 | 99.99532 |
| | | R5 | 6.17 | 1.5 | 5.83 | 4.67 | 99.99786 |
| | | R6 | 5.50 | 1.5 | 5.50 | 4.00 | 99.99000 |
| 60 | Oct. 5, 2020 | R1 | 4.83 | 1.5 | 4.50 | 3.33 | 99.95323 |
| | | R2 | 4.50 | 1.5 | 3.50 | 3.00 | 99.90000 |
| | | R3 | 4.17 | 1.5 | 3.83 | 2.67 | 99.78620 |
| | Oct. 12, 2020 | R4 | 4.50 | 2.17 | 4.17 | 2.33 | 99.99532 |
| | | R5 | 4.50 | 1.50 | 4.50 | 3.00 | 99.99000 |
| | | R6 | 4.50 | 1.50 | 4.50 | 3.00 | 99.99786 |

Log10 viral genome copy number/sample

| | | | | | | | |
|---|---|---|---|---|---|---|---|
| 10 | Oct. 12, 2020 | R1 | 8.26 | 6.89 | 8.25 | 1.37 | 95.74229 |
| | | R2 | 8.23 | 6.54 | 8.26 | 1.69 | 97.96647 |
| 30 | | R1 | 8.23 | 7.46 | 8.16 | 0.77 | 82.93884 |
| | | R2 | 8.19 | 6.76 | 8.23 | 1.43 | 96.26875 |
| 60 | | R1 | 8.15 | 6.70 | 8.17 | 1.45 | 96.44253 |
| | | R2 | 8.23 | 6.66 | 8.27 | 1.57 | 97.32023 |

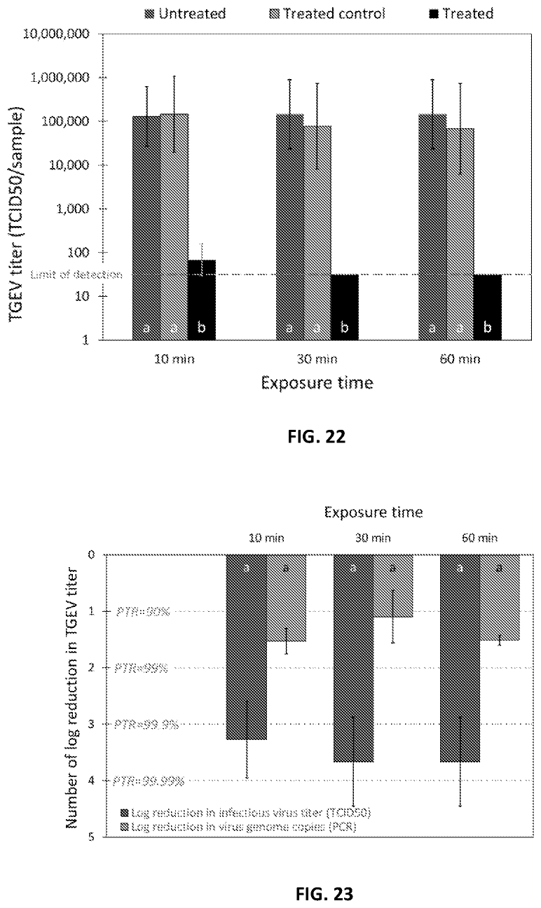

The titer of infectious TGEV particles recovered from face mask textile specimens after 10, 30, and 60 min contact times is shown in FIG. 22. At each time point, the first bar is untreated sample, the second bar is treated control samples, and the third bar is the treated samples. The columns are the geometric mean of 6 replicates. The error bars represent ±one geometric standard deviation. The scattered green line is the limit of detection. Same letters at each column base indicate geometric means that are not significantly different from one another at each contact time p≥0.05.

FIG. 23 is a graph of the Log₁₀ reduction in the infectious titer (first bar) and viral genome copies (second bar) after 10, 30, and 60 min contact times with Nylon-cotton textile specimens. The columns are the arithmetic mean and the error bars represent ±one standard deviation. Same letters at each column base indicate geometric means that are not significantly different from one another at each contact time p≥0.05. PTR=percentage of virus titer reduction.

The results show that both of the treated textiles (Nylon-cotton and face mask material) could neutralize more than 3 order of magnitude of the infectious TGEV (≥99.9%) within 10 min of contact in humid conditions in the presence of an organic load (in the form of FBS in virus suspension). The lower reduction in the number of TGEV genome copies indicates that the majority of the neutralized viral particles were inactivated by the impact of the textile active ingredients on the viral envelope and/or capsid proteins. The small fraction of viral genome that was reduced indicate that disintegration in the viral capsids occurred in approximately >90% of the viral particles during 10 min of contact with the treated textiles. The strong virucidal efficacy of the nanocomposite materials despite the presence of high protein organic load indicates that this efficacy will not be affected by the high protein content of human's sputum droplets in which viruses such as Covid-19 are shed.

### Example 7

Nanocomposite materials were created using polyester, silk and nylon/cotton textiles by immersing the textiles in a zinc nitrate, zinc acetate, zinc sulfate, and zinc chloride at an ideal concentration range of 0.1 to 0.75M for 30 minutes followed by heating in a conventional oven at 100° C. until dry. The nanocomposite materials were then exposed to a fungal strain of Candida Albicans per AATCC 30 method. The level of Candida Albicans was measured immediately after exposure (time zero) and after 24 hours of exposure to both the nanocomposite textiles as well as equivalent untreated textiles. The results are shown below in Tables 8-10, which includes the minimum, maximum and mean values for each sample at time 0 and at 24 hours. In all of the untreated textiles, there was an increase in the amount of fungus after 24 hours. In all of the treated textiles, there was a significant immediate reduction in the amount of fungus at time zero, followed by a complete elimination of fungus at 24 hours.

TABLE 8

| Material | Mean | Max | Min |
|---|---|---|---|
| Polyester untreated T0 | 4.20E+3 | 1.01E+2 | 9.88E+1 |
| Polyester treated T0 | 1.52E+3 | 1.02E+3 | 6.11E+2 |
| Polyester untreated T24 | 5.69E+5 | 2.52E+6 | 4.64E+5 |
| Polyester treated T24 | 0.00E+0 | 0.00E+0 | 0.00E+0 |

TABLE 9

| Material | Mean | Max | Min |
|---|---|---|---|
| Silk untreated T0 | 1.78E+3 | 3.59E+2 | 2.99E+2 |
| Silk treated T0 | 9.46E+2 | 2.82E+2 | 2.17E+2 |
| Silk untreated T24 | 4.22E+5 | 3.02E+6 | 3.70E+5 |
| Silk treated T24 | 0.00E+0 | 0.00E+0 | 0.00E+0 |

TABLE 10

| Material | Mean | Max | Min |
|---|---|---|---|
| Nylon/cotton untreated T0 | 4.61E+2 | 3.31E+2 | 1.93E+2 |
| Nylon/cotton treated T0 | 1.72E+2 | 2.32E+2 | 9.87E+1 |
| Nylon/cotton untreated T24 | 5.07E+4 | 2.58E+6 | 4.97E+4 |
| Nylon/cotton treated T24 | 0.00E+0 | 0.00E+0 | 0.00E+0 |

### Example 8

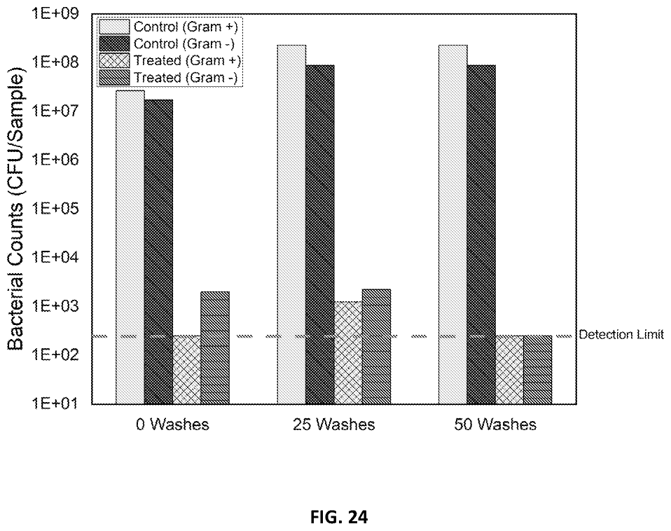

In another example, nanocomposite materials were fabricated at an industrial textile mill in the United States. A polyester/cotton blend fabric material was submerged in a treatment bath filled with the ionic precursor solution, as well as other finishing agents. Specifically, a zinc nitrate, zinc acetate, zinc sulfate, and zinc chloride at an ideal concentration range of 0.1 to 0.75M solution was used in combination with a solution containing a fabric softening agents, an optical brightener, and a soil release agent. The fabric was then passed through an industrial heating system and heated at 150° C. for approximately 3 minutes. The final fabric was then spooled up, and a sample was sent to Vartest Laboratories LLC for antiviral and antibacterial efficacy testing. Testing for antibacterial efficacy was conducted following the AATCC 100 protocol using Staphylococcus Aureus and Klebsiella Pneumoniae. The results are shown in the graph presented in FIG. 24. Antiviral efficacy was tested using the ISO 18184 testing protocol utilizing human coronavirus OC43.

Efficacy testing was performed on the nanocomposite materials as received, and then after 50 washes following a standardized washing methodology, AATCC TM96 specification VIc. Results are displayed in Table 11 below, in which percent indicates percent bacterial reduction measured in colony forming units per milliliter relative to a control.

TABLE 11

| | Staphylococcus Aureus | | Klebsiella Pneumoniae | | Human Coronavirus OC43 | |
|---|---|---|---|---|---|---|
| | Untreated | Treated | Untreated | Treated | Untreated | Treated |
| 0 Washes | 0% | 99.999% | 0% | 99.999% | 0% | 99.99% |
| 50 Washes | 0% | 99.999% | 0% | 99.999% | 0% | 99.8% |

As used herein, the terms “substantially” or “generally” refer to the complete or near complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially” free of or “generally” free of an element may still actually contain such element as long as there is no significant effect thereof.

In the foregoing description various embodiments of the invention have been presented for the purpose of illustration and description. They are not intended to be exhaustive or to limit the invention to the precise form disclosed. Obvious modifications or variations are possible in light of the above teachings. The embodiments were chosen and described to provide illustrations of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. An antimicrobial textile comprising:
a sheet substrate comprising a textile;
metal oxide nanoparticles;
wherein the nanoparticles are present as a nanocomposite on the surface of and within the sheet substrate.

2. The antimicrobial textile of claim 1 wherein the antimicrobial textile is configured to be worn on a body of a user.

3. The antimicrobial textile of claim 2 wherein the antimicrobial textile is personal protective equipment.

4. The antimicrobial textile of claim 3 wherein the personal protective equipment comprises a multilayer face mask and wherein the sheet substrate comprises one layer of the face mask.

5. The antimicrobial textile of claim 3 wherein the personal protective equipment comprises clothing.

6. The antimicrobial textile of claim 2 further comprising an adhesive layer.

7. The antimicrobial textile of claim 6 wherein the personal protective equipment comprises a bandage.

8. The antimicrobial textile of claim 1 wherein the antimicrobial textile comprises a feminine hygiene product.

9. The antimicrobial textile of claim 1 wherein the metal oxide comprises zinc oxide.

10. The antimicrobial textile of claim 1 wherein the antimicrobial textile comprises a furniture upholstery.

11. The antimicrobial textile of claim 1 wherein the antimicrobial textile comprises a surface cleaning product.

12. The antimicrobial textile of claim 11 wherein the surface cleaning product comprises a mop, sponge, rag or towel.

13. The antimicrobial textile of claim 1 wherein the antimicrobial or antiviral textile comprises an article of bedding.

14. An antimicrobial face mask comprising:
a multilayer sheet portion configured to cover a nose and mouth of a user, one or more of the sheets comprising a metal oxide textile nanocomposite; and
straps configured for attachment of the mask to a user's head.

15. The antimicrobial face mask of claim 14 wherein the metal oxide comprises zinc oxide.

16. A method of making an antimicrobial textile comprising a nanocomposite sheet, the nanocomposite sheet produced by the method of:
applying a metal salt solution to a textile to diffuse the metal salt into the textile, the textile comprising a surface and interior fibers;
drying the textile with the applied metal salt solution to bind the metal salt to the surface of and the interior fibers of the textile by forming a nanocomposite of metal nanoparticles or nanostructures in situ.

17. The method of claim 16 wherein drying the textile comprises heating the sheet.

18. The method of claim 16 wherein the metal salt comprises zinc oxide.

19. The method of claim 16 further comprising incorporating the nanocomposite sheet into a wearable article.

20. The method of claim 19 wherein the wearable article comprises an article of personal protective equipment.
